(19) United States
(12) Patent Application Publication
(10) Pub. No.: US 2025/0177027 A1
(43) Pub. Date: Jun. 5, 2025

(54) APPARATUS AND METHOD FOR PROTECTING BIOLOGICAL TISSUE DURING CRYOTHERAPY

(71) Applicant: BioProtect Ltd., Tzur Yigal (IL)

(72) Inventor: Shaul Shohat, Kfar HaOranim (IL)

(21) Appl. No.: 18/843,128

(22) PCT Filed: May 11, 2023
(86) PCT No.: PCT/IB2023/054890

§ 371 Date: Aug. 30, 2024

Related U.S. Application Data
(60) Provisional application No. 63/341,484, filed on May 13, 2022.

(51) Int. Cl. A61B 18/02; A61B 18/00

(52) U.S. Cl. CPC A61B 18/02; A61B 2018/00023; A61B 2018/00547; A61B 2018/00577; A61B 2018/00642; A61B 2018/00714; A61B 2018/00791

(57) ABSTRACT

In a method for safeguarding a subject's rectum during a cryoablation therapy session targeting the subject's prostate, a deflated tissue-dissection balloon is inserted into a perirectal space between the prostate and the rectum, e.g., subsequent to introduction of one or more cryoprobes to the prostate. The balloon is inflated with a thermal fluid so as to create a thermal-separation barrier between the prostate and a rectal wall, the thermal fluid is circulated, at least during cryoablation therapy session, by aspirating, from the balloon, thermal fluid cooled by the targeted cryoablation therapy, and, subsequently or in parallel, reinflating the balloon with thermal fluid that is warmer than the aspirated fluid.

### CROSS-REFERENCE TO RELATED APPLICATION

This application claims the benefit of U.S. Patent Application No. 63/341,484 entitled “APPARATUS AND METHOD FOR PROTECTING BIOLOGICAL TISSUE DURING CRYOTHERAPY” and filed on May 13, 2022, which is incorporated by reference herein in its entirety.

### FIELD OF THE INVENTION

The present invention relates to a balloon spacer apparatus and in particular to its use in safeguarding a subject's rectum during a cryoablation therapy session targeting the subject's prostate.

### BACKGROUND

Inter-tissue and inter-organ spacers are often used for creating and occupying a dissected tissue space in human subject. Typically such spacers are used to distance a healthy tissue or organ from another tissue or organ that is targeted for a treatment such as, for example, a radiation treatment. The tissue dissection process and spacer placement can reduce the exposure of the healthy tissue to the potentially negative effects of the treatment. The spacers are often introduced into the tissue spaces while the surgeon is guided by ultrasound imaging. Targeted cryoablation therapy is used to destroy portions of an afflicted prostate, and collateral damage from freezing can occur to adjacent rectal tissue. Thus there is a need for a solution to providing thermal protection to a rectum during a cryoablation therapy session targeting the subject's prostate.

### SUMMARY

Embodiments of the present invention relate to safeguarding (or, equivalently, protecting) a subject's rectum during a cryoablation therapy session targeting the subject's prostate.

A method is disclosed, according to embodiments, for safeguarding a subject's rectum during a cryoablation therapy session targeting the subject's prostate. The method comprises: inserting a deflated tissue-dissection balloon into a perirectal space between the prostate and the rectum; inflating the balloon with a thermal fluid so as to create a thermal-separation barrier between the prostate and a rectal wall; and circulating the thermal fluid by aspirating, from the balloon, thermal fluid cooled by the targeted cryoablation therapy, and subsequently reinflating the balloon with thermal fluid that is warmer than the aspirated fluid.

In some embodiments, the inserting is carried out subsequent to introduction of one or more cryoprobes to the prostate.

In some embodiments, the circulating is carried out subsequent to introduction of one or more cryoprobes to the prostate.

In some embodiments, the reinflating can be carried out subsequent to the aspirating. In some embodiments, the aspirating and reinflating can be carried out in parallel. In some embodiments, the aspirating and reinflating can be carried out simultaneously.

In some embodiments, the circulating can be effective to avoid exposing the rectum to a temperature below 20° C., or below 15° C., or below 10° C. In some embodiments, any method can additionally comprise: aspirating the thermal fluid from the balloon through a fluid conveyance so as to substantially deflate the balloon, and withdrawing the fluid conveyance without removing the balloon from the perirectal space. In some such embodiments, the balloon can be biodegradable in situ. In some embodiments, any method can additionally comprise: aspirating the thermal fluid from the balloon so as to substantially deflate the balloon, and removing the balloon from the perirectal space.

In some embodiments, the thermal fluid can include an aqueous saline solution having an NaCl concentration between 0.05 and 0.5 mol/kg.

In some embodiments, the reinflating of the balloon is with a thermal fluid having a temperature that is between 5° C. and 30° C. warmer than the aspirated fluid, or between 10° C. and 30° C. warmer, or between 10° C. and 20° C. warmer. In some embodiments, the thermal fluid for the reinflation can be heated before the reinflation. In some embodiments, the thermal fluid for reinflation can be at least partially the same fluid aspirated from the balloon, and in other embodiments, the thermal fluid for reinflation can fluid that was not aspirated from the balloon, or that was aspirated from the balloon in a previous circulation cycle.

In some embodiments, a temperature of the aspirated fluid can be at least 10° C., or at least 15° C., or at least 20° C. In some embodiments, the temperature of the warmer fluid can be at least 20° C. and not more than 42° C.

In some embodiments, the circulating can be carried out at least twice during the cryoablation therapy session. In some embodiments, the circulating can include at least 2 cycles of aspirating and reinflating during the cryoablation therapy session, or at least 5 cycles, or at least 10 cycles.

In some embodiments, the circulating can be initiated and/or repeated in response to receiving a temperature measurement of the thermal fluid. In some embodiments, the circulating can be initiated and/or repeated in response to receiving a temperature measurement of rectal tissue. In some embodiments, the circulating can be initiated and/or repeated automatically in response to the temperature measurement.

In some embodiments, the circulating can be initiated and/or repeated according to a programmed schedule.

In some embodiments, it can be that at least one aspiration of the cooled thermal fluid is a partial aspiration, and a respective subsequent reinflation is from a state of partial inflation. In some such embodiments, the state of partial inflation can be between 10% and 80% by volume of full inflation, or between 40% and 70% by volume of full inflation.

In some embodiments, it can be that an average temperature of the thermal fluid in the balloon does not go lower than 14° C., or lower than 15° C., or lower than 16° C., or lower than 17° C., or lower than 18° C., or lower than 19° C., or lower than 20° C., or lower than 21° C., or lower than 22° C., or lower than 23° C., or lower than 24° C., during the cryoablation therapy session.

In some embodiments, it can be that a temperature of rectal tissue in contact with the balloon in an inflated state does not go below 14° C., or below 15° C., or lower than 16° C., or lower than 17° C., or lower than 18° C., or lower than 19° C., or lower than 20° C., or lower than 21° C., or lower than 22° C., or lower than 23° C., or lower than 24° C., during the cryoablation therapy session.

According to embodiments of the present invention, an apparatus for use in safeguarding a subject's rectum during a cryoablation therapy session targeting the subject's prostate comprises: an inflatable spacer balloon configured to hold a quantity of a thermal fluid and to undergo multiple in situ inflation-deflation cycles; and a circulation assembly configured to be placed in fluid communication with the balloon and adapted to circulate the thermal fluid by aspirating cooled thermal fluid from the balloon and reinflating the balloon with thermal fluid that is warmer than the aspirated fluid.

In some embodiments, the apparatus can additionally comprise a medical dilator assembly for an ultrasound-guided tissue dissection, the medical dilator assembly including a lumen for passing the balloon therethrough, in an uninflated state, into a perirectal space between the prostate and the rectum.

In some embodiments, the circulation assembly can comprise a plurality of fluid conveyances, wherein a first fluid conveyance is for aspiration and a second fluid conveyance is for reinflation. In some embodiments, the circulation assembly can comprise a plurality of fluid conveyances, wherein a first fluid conveyance is configured to convey the thermal fluid at a pressure above an ambient pressure, and a second fluid conveyance is configured to convey the thermal fluid at a pressure below the ambient pressure. In some embodiments, the circulation assembly can comprise a fluid conveyance in fluid communication with multiple other fluid conveyances. In some embodiments, the circulation assembly can comprise a fluid conveyance comprising multiple internal lumens, wherein a first lumen is for aspiration and a second lumen is for reinflation.

In some embodiments, the circulation assembly can comprise a fluid conveyance comprising multiple internal lumens, wherein a first lumen is configured to convey the thermal fluid at a pressure above an ambient pressure, and a second fluid lumen is configured to convey the thermal fluid at a pressure below the ambient pressure.

In some embodiments, the circulation assembly can additionally comprise a temperature sensor for measuring an external temperature of the balloon. In some embodiments, the circulation assembly can additionally comprise a temperature sensor for measuring a temperature of thermal fluid within the balloon. In some embodiments, the circulation assembly can additionally comprise a temperature sensor for measuring a temperature of thermal fluid aspirated from the balloon. In some embodiments, the circulation assembly can additionally comprise a temperature sensor for measuring a temperature of a wall of the rectum. In some embodiments, the circulation assembly can additionally comprise a temperature sensor in fluid communication with the heated thermal fluid.

In some embodiments, the circulation assembly can additionally comprise a circulation controller programmed to initiate a circulating of the thermal fluid in response to a temperature measurement. In some embodiments, the circulation assembly can additionally comprise a circulation controller programmed to initiate a circulating of the thermal fluid based on a programmed schedule. In some embodiments, the circulation assembly can additionally comprise a circulation controller programmed to initiate a circulating of the thermal fluid based on avoiding exposing the rectum to a temperature below 20° C., or below 15° C., or below 10° C.

In some embodiments, the balloon can be biodegradable in situ.

### BRIEF DESCRIPTION OF THE DRAWINGS

The invention will now be described further, by way of example, with reference to the accompanying drawings, in which the dimensions of components and features shown in the figures are chosen for convenience and clarity of presentation and not necessarily to scale. In the drawings:

FIG. 1 shows an exemplary spacer balloon in an inflated state, according to embodiments of the present invention.

FIG. 2A is a schematic illustration of an apparatus comprising a spacer balloon and a circulation assembly, according to embodiments of the present invention.

FIGS. 2B, 2C and 2D show a detail of the apparatus of FIG. 2A, according to embodiments of the present invention.

FIG. 3 is a schematic illustration of an apparatus comprising a spacer balloon and a circulation assembly, according to embodiments of the present invention.

FIG. 4 shows a block diagram of a circulation controller, according to embodiments of the present invention.

FIG. 5 is a block diagram of a circulation assembly according to embodiments of the present invention.

FIG. 6 is a schematic perspective view of a dilator assembly, according to embodiments of the present invention.

FIGS. 7A, 7B, 7C, 7D, 7E, and 7F are schematic illustrations of an exemplary method of inserting a spacer balloon in a perirectal space.

FIGS. 8, 9 and 10 show flowcharts of methods and method steps for safeguarding a subject's rectum during a cryoablation therapy session targeting the subject's prostate, according to embodiments of the present invention.

### DETAILED DESCRIPTION OF THE ILLUSTRATED EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are generally used to designate like elements. Subscripted or letter-modified reference numbers (e.g., 100_{A}) are used to designate multiple separate appearances of elements in a single drawing, e.g. 100_{A} is a single appearance (out of a plurality of appearances) of element 100.

For convenience, in the context of the description herein, various terms are presented here. To the extent that definitions are provided, explicitly or implicitly, here or elsewhere in this application, such definitions are understood to be consistent with the usage of the defined terms by those of skill in the pertinent art(s). Furthermore, such definitions are to be construed in the broadest possible sense consistent with such usage. A “spacer balloon”, as the term is used herein, is an implantable, inflatable balloon used for tissue dissection. Apparatuses and methods for dissection of one tissue from another, or one organ from another, are particularly applicable to providing thermal protection to one of the tissues. disclosed. The dissection can be accomplished by delivering a spacer, such as an inflatable balloon, to reside between the tissues or organs until such time that the spacer biodegrades and/or is removed. Such spacers are known to be useful in cases where physical separation between adjacent tissues and/or organs is desirable, for example, to safeguard one tissue or organ from effects of a cryoablation treatment to the second tissue or organ. Examples of such spacers can be found in co-pending U.S. patent application Ser. No. 16/273,030, published as US Patent Publication No. US20190239849A1, which is incorporated herewith by reference in its entirety.

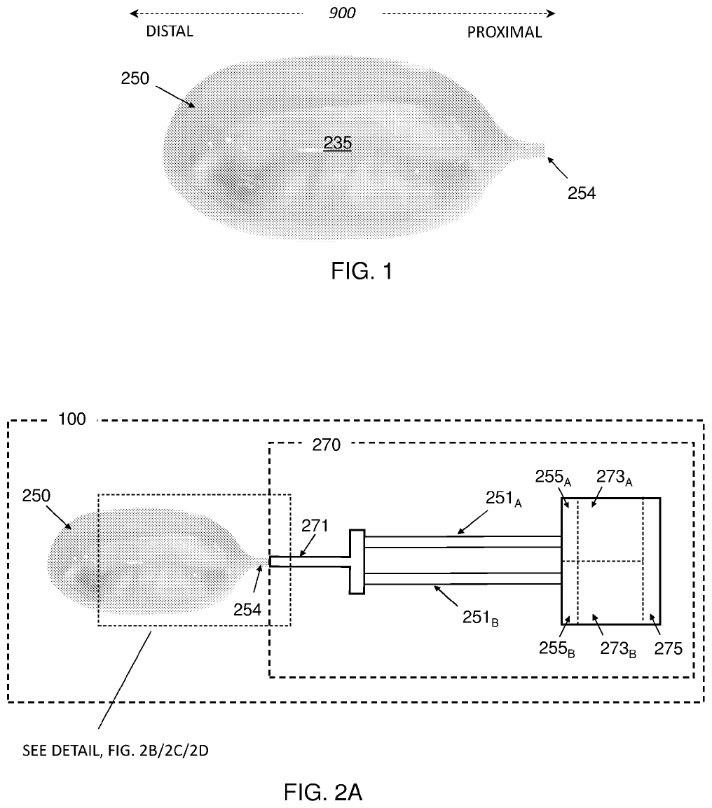

Referring now to the figures and in particular to FIG. 1, a spacer balloon 250 has an internal volume at full inflation (as shown) of at least 5 ml, or at least 10 ml, or at least 15 ml, or at least 20 ml, or at least 25 ml, or at least 30 ml, or at least 35 ml, or at least 40 ml. In embodiments, the balloon 250 has an internal volume of not more than 50 ml, or not more than 40 ml, or not more than 30 ml, or not more than 25 ml, or not more than 20 ml, or not more than 15 ml. The balloon 250, as shown in FIG. 1 includes an aperture 254 for inflation of the balloon 250, e.g., with a thermal fluid 235.

Arrow 900 in FIG. 1 indicates the relative directions of ‘distal’ and ‘proximal’ as used herein. This convention, with distal being leftward and proximal being rightwards, is maintained throughout the figures.

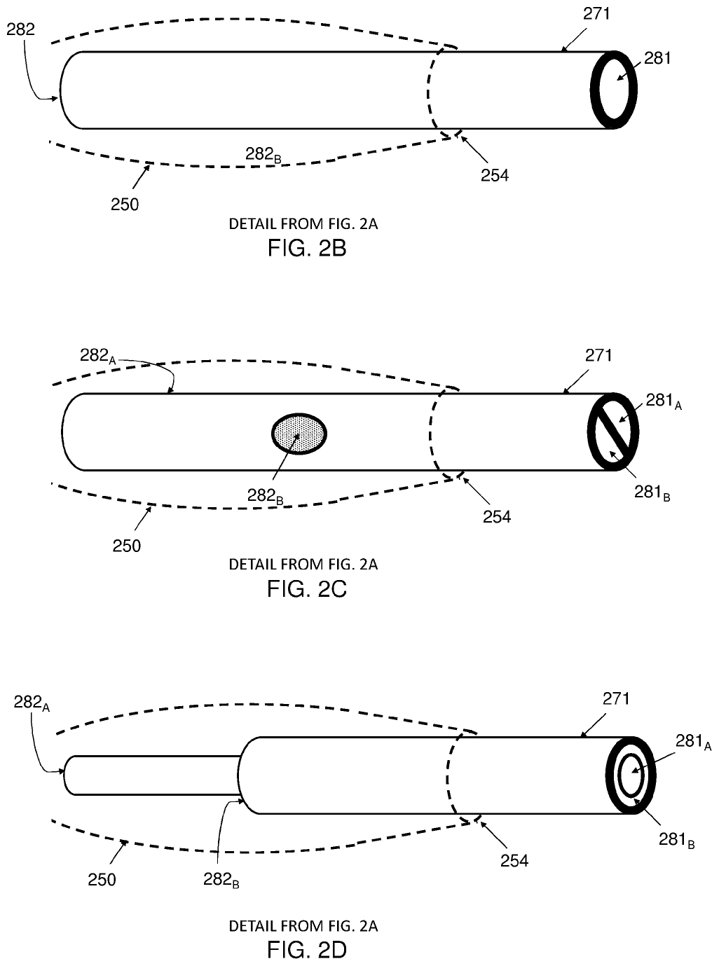

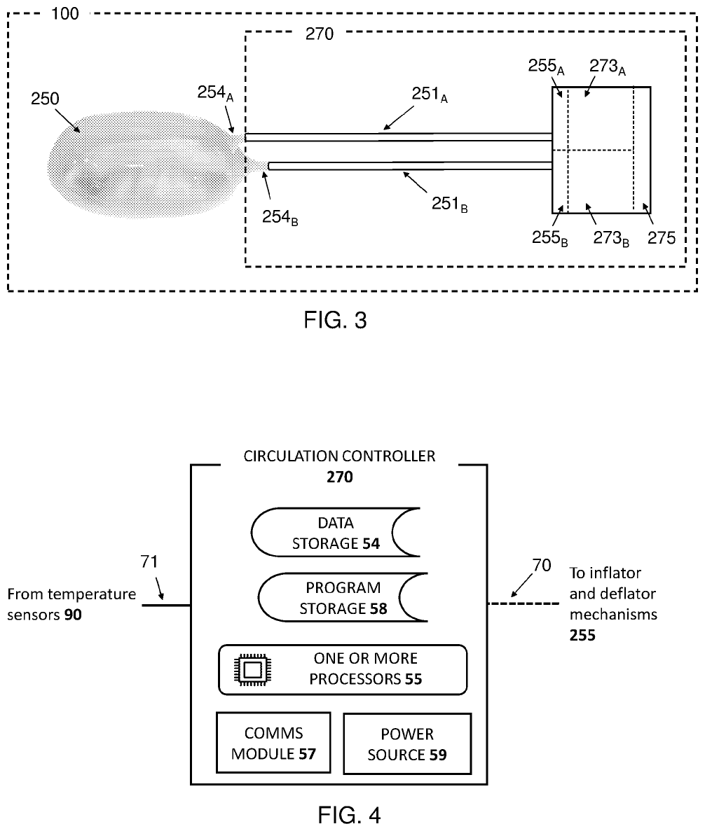

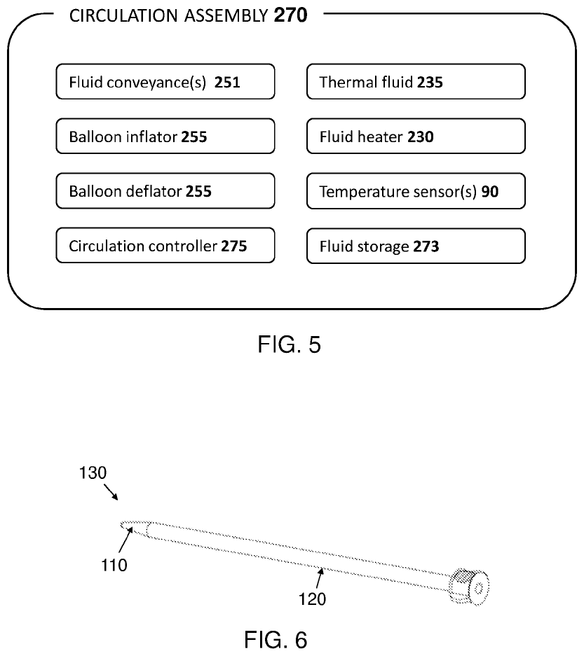

Reference is made to FIGS. 2A, 2B, 2C, 2D and 3, which illustrate respective non-limiting examples of an apparatus for use in safeguarding a subject's rectum during a cryoablation therapy session targeting the subject's prostate. An apparatus 100 includes a spacer balloon 250 and a circulation assembly 270, illustrated in FIGS. 2A, 2B, 2C, 2D and 3 in an assembled state.

FIG. 2A shows a balloon 250 that is the same as or similar to the balloon of FIG. 1. The circulation assembly 270 includes a T-connector or splitter 271 for connecting two fluid conveyances 251_{A}, 251_{B} to the single aperture 254 of the balloon 250. In embodiments, a first fluid conveyance 251_{A} can be used for aspiration of fluid 235 and a second fluid conveyance 251_{B} can be used for reinflation of the balloon 250.

The splitter 271, which may be of any practical design, i.e., not necessarily a T-connector as shown is for connecting a single aperture 254 of the balloon 250 with a circulation assembly 270 that has two mechanisms, e.g., a separate inflator 255_{A} and deflator 255_{B}. FIG. 2B shows a detail of FIG. 2A, in which the distal portion of the splitter 271 enters the aperture 254 of the balloon 250 as a single fluid conveyance comprising a single internal lumen 281 having a single distal aperture 282 within the balloon 250. This configuration is particularly useful in embodiments in which each reinflation of the balloon 250 is subsequent to a corresponding deflation/aspiration of thermal fluid 235.

In some embodiments, the splitter 271 can include a single fluid conveyance that enters the balloon 250 via the single aperture 254 but that includes two separate internal lumens 281_{A}, 281_{B}, having respective distal apertures 282_{A}, 282_{B} within the balloon 250, as shown in the non-limiting illustrative examples of FIGS. 2C and 2D. In yet another non-limiting example (not shown) the single fluid conveyance that enters the balloon via the single aperture 254 can split into two fluid conveyances inside the balloon 250. This arrangement can be useful either for embodiments in which each reinflation of the balloon 250 is subsequent to a corresponding deflation/aspiration of thermal fluid 235, or for embodiments in which each aspiration and its corresponding reinflation are carried out in parallel or simultaneously. “In parallel” as used here can include any one of “simultaneously”, “overlappingly,” or “alternatingly”.

As shown in FIGS. 2C and 2D, The distal apertures 282_{A}, 282_{B} can be directed toward different internal portions of the balloon 250 so as to improve the circulation, for example when one portion of the fluid to be aspirated is colder than another portion.

FIG. 3, in contrast to FIG. 2A, shows a balloon 250 according to alternative embodiments in which the balloon 250 comprises two apertures 254_{A}, 254_{B}, where one of the apertures 254A is configured for a flow therethrough of a fluid 235 for inflating the balloon 250, and the second aperture 254_{B} is configured for a reverse flow therethrough of the fluid 235 for aspirating the fluid from the balloon 250. In embodiments, it does not matter which of 254_{A}, 254_{B} is used for inflation and which is used for deflation.

This arrangement can also be useful either for embodiments in which each reinflation of the balloon 250 is subsequent to a corresponding deflation/aspiration of thermal fluid 235, or for embodiments in which each aspiration and its corresponding reinflation are carried out in parallel or simultaneously.

The circulation assembly 270 of FIGS. 2A and 3 further includes inflator/deflator mechanisms 255_{A}, 255_{B}. Examples of inflator and deflator mechanisms include, and not exhaustively: pumps, valves and/or syringes. Optional fluid storage volumes 273_{A}, 273_{B} are provided for storing excess fluid 235 and/or for heating the aspirated fluid 235. To that end, a heater 230 can be provided, either for heating fluid 235 within a storage volume 273 or within a fluid conveyance 251.

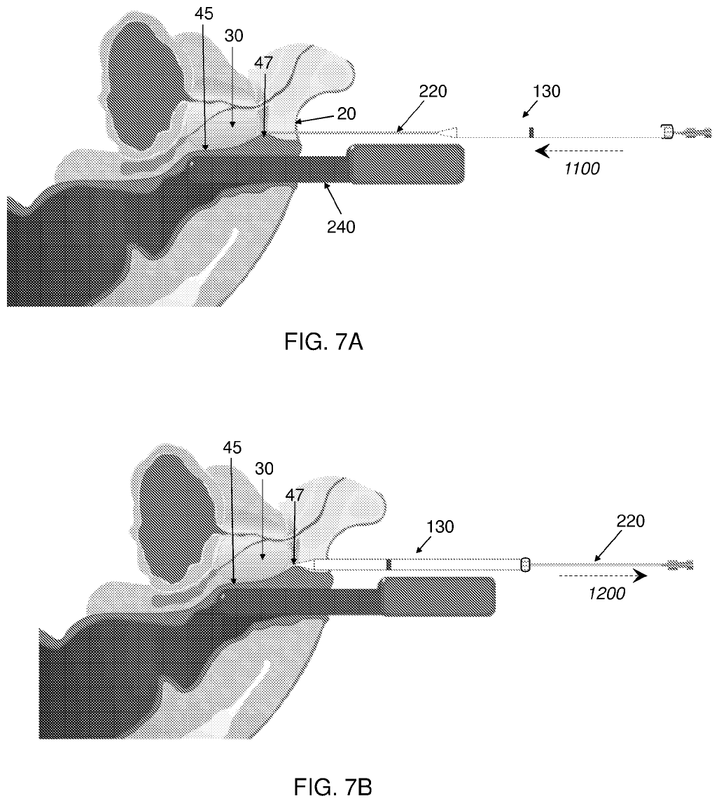

Referring now to FIG. 4, a non-limiting example of a circulation controller 275 according to embodiments is illustrated schematically to show selected components. The exemplary controller 275 of FIG. 4 includes one or more computer processors 55, a computer-readable storage medium 58, a communications module 57, and a power source 59. The computer-readable storage medium 58 can include transient and/or long-term storage, and can include one or more storage units, all in accordance with desired functionality and design choices. The storage 58 can be used for any one or more of: storing program instructions, in firmware and/or software, for execution by the one or more processors 55 of the control system 275. In embodiments, the stored program instructions include program instructions for controlling the circulation system 270 for the purpose of safeguarding a subject's rectum during a cryoablation therapy session targeting the subject's prostate 100. Data storage 54, if separate from storage 58, can be provided for historical data. In some embodiments, the two storage modules 54, 58 form a single module. The communications module 57 is configured to establish communications links, e.g., via communication arrangements 70 with inflator and deflator mechanisms 255 and/or via communication arrangements 71 for receiving measurements from temperature sensor(s) 90. In some embodiments, a control system 275 does not necessarily include all of the components shown in FIG. 4. The terms “communications arrangements” or similar terms such as “communications links” as used herein mean any wired connection or wireless connection via which data communications can take place. Non-limiting and non-exhaustive examples of suitable technologies for providing communications arrangements include any short-range point-to-point communication system such as IrDA, RFID (Radio Frequency Identification), TransferJet, Wireless USB, DSRC (Dedicated Short Range Communications), or Near Field Communication; wireless networks (including sensor networks) such as: ZigBee, EnOcean; Wi-fi, Bluetooth, TransferJet, or Ultra-wideband; and wired communications bus technologies such as. CAN bus (Controller Area Network, Fieldbus, FireWire, HyperTransport and InfiniBand.

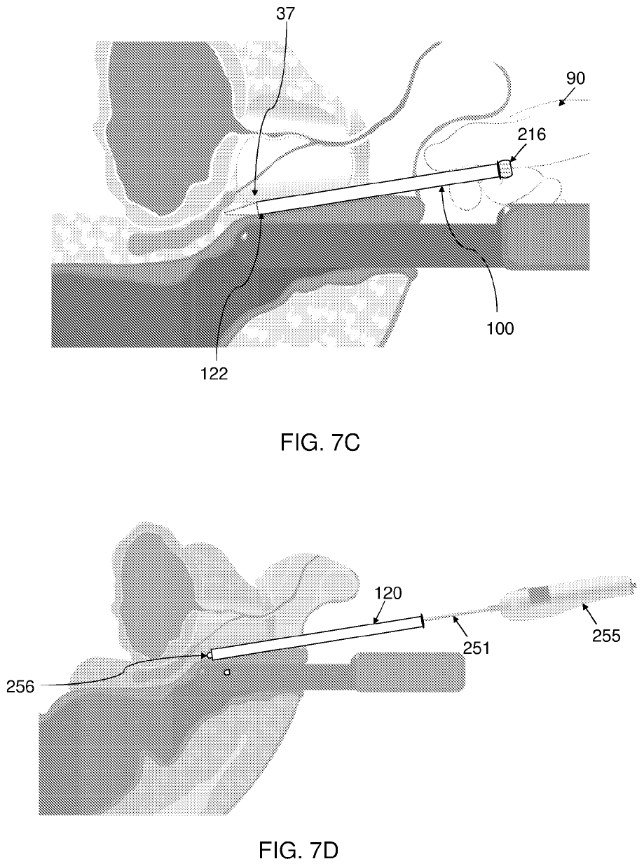

The block diagram of FIG. 5 shows, and not exhaustively, major components of a circulation system 270, including fluid conveyance(s) 251, balloon inflator and deflator 255, circulation controller 275, and optionally: thermal fluid 235, a fluid heater 230, one or more temperature sensors 90, and fluid storage compartments or containers 273.

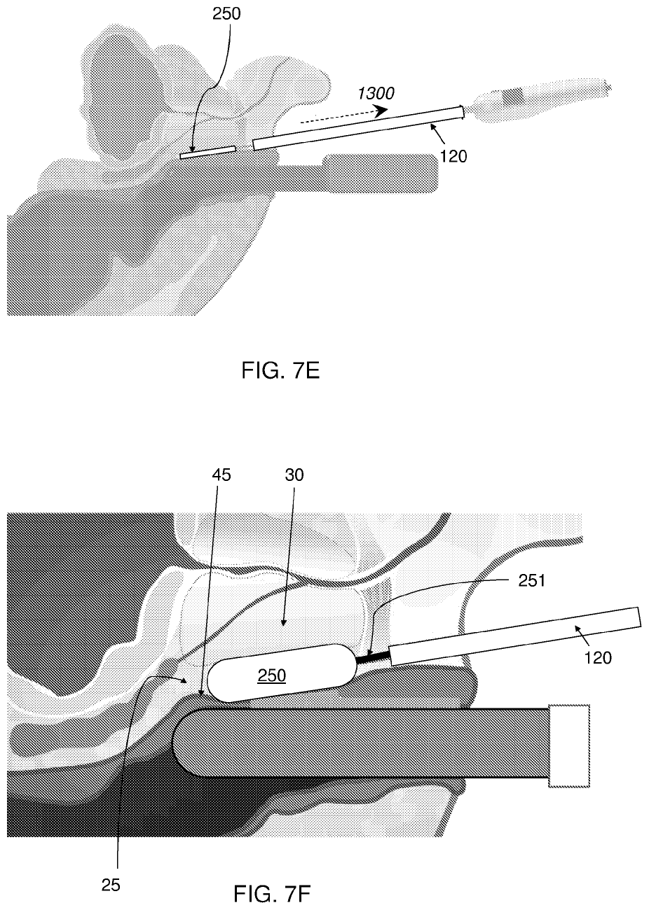

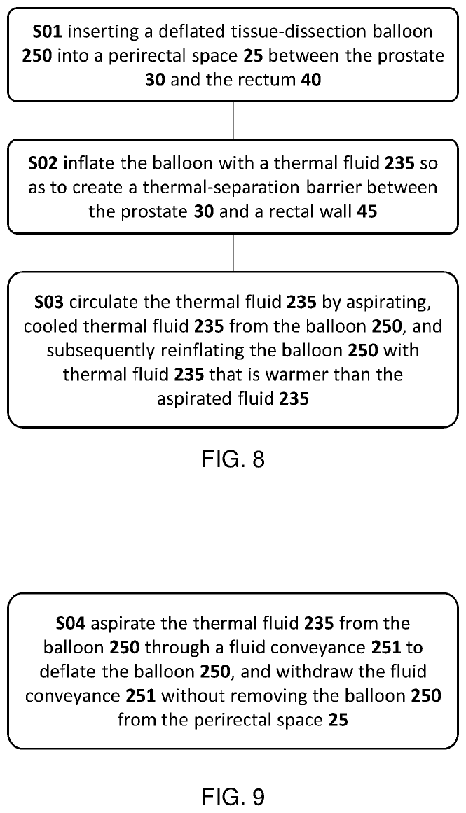

FIGS. 6 and 7A-E relate to an exemplary method for inserting a spacer balloon 250 into a perirectal space 25 between the prostate 30 and the rectum 40.

FIG. 6 shows a dilator assembly 130 comprising a dilator tip 110 and a dilator sheath or body 120, the use of which is described in the following paragraphs.

FIGS. 7A-7E illustrate an exemplary use case wherein methods disclosed herein are applied to the dissection of a subject's prostate 30 from the subject's rectum 40 by inserting a spacer balloon 250 therebetween, according to embodiments. Those skilled in the art will understand that there may be other acceptable methods for performing the inserting.

As seen in FIG. 7A, a transrectal ultrasound (TRUS) probe 240 has already been deployed for guiding the procedure. A dilator assembly 130 surrounds a guide needle 220, which interiorly traverses the dilator assembly 130, and is advanced in FIG. 7A, in the direction indicated by arrow 1100 over the guide needle 220. The needle has already been advanced to a first surface 47 of the rectal wall 45. In FIG. 7B, the dilator assembly 130 has been advanced along the guide needle 220 and inserted through an incision in the subject's perineum 20 until the distal tip 110 of the dilator assembly 130 reaches said first surface 47 of the rectal wall 45. As indicated by arrow 1200, the guide needle 220 is proximally withdrawn once the distal tip of the dilator assembly 100 has reached said first surface 47.

FIG. 7C shows the dilator assembly already advanced, e.g., by a user pushing the dilator assembly 130 and/or adjusting the entry angle of the dilator assembly 130, until the dilator assembly 130 has reached a target location, e.g., the base 37 of the prostate 30.

Referring now to FIG. 7D, the dilator 130 has been withdrawn from the dilator sheath 120 and the balloon 250, e.g., in a folded-up or rolled-up state, is inserted through the dilator-sheath 120 until a distal end 256 is aligned with the distal end 122 of the sheath 120. The balloon is connected to the inflator 255 by fluid conveyance 251. In FIG. 7E, it can be seen that the dilator sheath 120 is proximally withdrawn, as indicated by arrow 1300, to expose the folded-up or rolled-up uninflated balloon 250. FIG. 7F shows schematically that the balloon 250 has been inflated, to a desired thickness between the prostate 30 and the rectal wall 45, using thermal fluid 235 (not shown) injected by the balloon inflator 255 through the fluid conveyance 251.

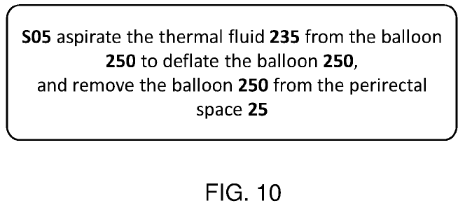

FIG. 8 shows a flowchart of a method for safeguarding a subject's rectum 40 during a cryoablation therapy session targeting the subject's prostate 30. The method can employ any of the apparatuses and devices disclosed in the various embodiments. As shown in the flowchart of FIG. 8, the method comprises at least Steps S01, S02 and S03:

Step S01: inserting a deflated tissue-dissection balloon 250 into a perirectal space 25 between the prostate 30 and the rectum 40. Some sub-steps of an exemplary method for carrying out Step S01, i.e., inserting the balloon 250, are shown schematically in FIGS. 7A-7E. In some embodiments, Step S01 is carried out subsequent to and contingent upon the introduction of one or more cryoprobes to the prostate for initiating the targeted cryoablation therapy session.

Step S02: inflating the balloon with a thermal fluid 235, as shown schematically in FIG. 7F, so as to create a thermal-separation barrier between the prostate 30 and a rectal wall 45.

Step S03 circulating the thermal fluid 235 by aspirating, cooled thermal fluid 235 from the balloon 250, and reinflating the balloon 250 with thermal fluid 235 that is warmer than the aspirated fluid 235. In some embodiments, Step S03 is carried out subsequent to and contingent upon the introduction of one or more cryoprobes to the prostate for initiating the targeted cryoablation therapy session. In some embodiments, the reinflating is carried out subsequent to the aspirating. In some embodiments, the aspirating and reinflating is carried out in parallel. In some embodiments, the aspirating and reinflating is carried out simultaneously.

Reinflating the balloon 250 is conceptually the same as the original inflation shown in FIG. 7F. Both the aspirating of the fluid 235 from the balloon 250 and the reinflating of the balloon can use any one of the circulation assemblies 270 of FIG. 2, 3 or 5.

In some embodiments, the reinflating of the balloon 250 is with thermal fluid 235 having a temperature that is between 5° C. and 30° C. warmer than the aspirated fluid 235, or between 10° C. and 30° C. warmer, or between 10° C. and 20° C. warmer. In some embodiments, the temperature of the aspirated fluid is at least 10° C., or at least 15° C., or at least 20° C. In some embodiments, the temperature of the warmer fluid 235 is at least 20° C. and not more than 42° C. In some embodiments, the aspirated fluid 235 is heated before reinflation of the balloon 250. In some embodiments, the thermal fluid 235 includes an aqueous saline solution 246 having an NaCl concentration between 0.05 and 0.5 mol/kg.

In some embodiments, Step S03 is carried out in a manner that avoids exposing the rectum 40, including the rectum wall 45 nearest the prostate, to a temperature below 20° C., or below 15° C., or below 10° C. According to embodiments, a temperature of rectal tissue in contact with the balloon in an inflated state does not go below 14° C., or below 15° C., or lower than 16° C., or lower than 17° C., or lower than 18° C., or lower than 19° C., or lower than 20° C., or lower than 21° C., or lower than 22° C., or lower than 23° C., or lower than 24° C., during the cryoablation therapy session. According to embodiments, an average temperature of the thermal fluid in the balloon does not go lower than 14° C., or lower than 15° C., or lower than 16° C., or lower than 17° C., or lower than 18° C., or lower than 19° C., or lower than 20° C., or lower than 21° C., or lower than 22° C., or lower than 23° C., or lower than 24° C., during the cryoablation therapy session.

In some embodiments, Step S03 is carried out at least twice during the cryoablation therapy session. In some embodiments, the Step S03 includes at least 2 cycles of aspirating and reinflating during the cryoablation therapy session, or at least 5 cycles, or at least 10 cycles. In some embodiments, at least one aspiration of the cooled thermal fluid 235 is a partial aspiration, and a respective subsequent reinflation is from a state of partial inflation. The state of partial inflation can be between 10% and 80% by volume of full inflation, or between 40% and 70% by volume of full inflation.

In some embodiments, the circulating of Step S03 is initiated and/or repeated in response to receiving a temperature measurement of the thermal fluid. In some embodiments, the circulating of Step S03 is initiated and/or repeated in response to receiving a temperature measurement of rectal tissue. In some embodiments, the circulating of Step S03 is initiated and/or repeated automatically in response to a temperature measurement. In some embodiments, the circulating of Step S03 is initiated and/or repeated according to a programmed schedule.

In some embodiments, Step S01 is carried out at a first time, and Steps S02 and S03 are carried out at a second time. In some embodiments, Step S01 is carried out once, and Steps S02 and S03 are carried out multiple times in conjunction with multiple cryoablation therapy sessions.

In some embodiments, the method additionally comprises Step S04, as shown in the flowchart of FIG. 9:

Step S04: aspirating the thermal fluid 235 from the balloon 250 through a fluid conveyance 251 to deflate the balloon 250, and withdraw the fluid conveyance 251 without removing the balloon 250 from the perirectal space 25.

In some embodiments, the method additionally comprises Step S05, as shown in the flowchart of FIG. 10:

Step S05: aspirating the thermal fluid 235 from the balloon 250 to deflate the balloon 250, and remove the balloon 250 from the perirectal space 25. In some embodiments, the balloon is biodegradable in situ.

In embodiments, a clinical implementation of the method in connection with a cryotherapy session can carry out either one of Step S05, which includes removing the balloon 250 after the therapy session, and Step S04, which includes leaving the balloon 250 in situ, where it can biodegrade. In some embodiments, however, the method can include Step S04 in connection with a first cryotherapy session and can include Step S05 in connection with a second cryotherapy session—i.e., leaving the balloon 250 in place after the first session in anticipation of a subsequent session, after which the balloon 250 may be removed if desired. In some embodiments, the decision of whether to remove the balloon 250 (Step S05) or leave it in place (Step S04 can be a case-by-case decision made by the clinician.

Although the disclosed inventive concepts include those defined in the attached claims, it should be understood that the inventive concepts can also be defined in accordance with the following embodiments.

In addition to the embodiments of the attached claims and the embodiments described above, the following numbered embodiments are also innovative.

Embodiment 1 is a method of safeguarding a subject's rectum during a cryoablation therapy session targeting the subject's prostate, the method comprising:
inserting a deflated tissue-dissection balloon into a perirectal space between the prostate and the rectum;
inflating the balloon with a thermal fluid so as to create a thermal-separation barrier between the prostate and a rectal wall; and
circulating the thermal fluid by aspirating, from the balloon, thermal fluid cooled by the targeted cryoablation therapy, and reinflating the balloon with thermal fluid that is warmer than the aspirated fluid.

Embodiment 2 is the method of embodiment 1, wherein the inserting is carried out subsequent to an introduction of one or more cryoprobes to the prostate.

Embodiment 3 is the method of embodiment 1, wherein the circulating is carried out subsequent to introduction of one or more cryoprobes to the prostate.

Embodiment 4 is the method of any one of embodiments 1-3, wherein the reinflating is carried out subsequent to the aspirating.

Embodiment 5 is the method of any one of embodiments 1-3, wherein the aspirating and reinflating are carried out in parallel.

Embodiment 6 is the method of any one of embodiments 1-3, wherein the aspirating and reinflating are carried out simultaneously.

Embodiment 7 is the method of any one of embodiments 1-6, wherein the circulating is effective to avoid exposing the rectum to a temperature below 20° C., or below 15° C., or below 10° C.

Embodiment 8 is the method of any one of embodiments 1-7, additionally comprising: aspirating the thermal fluid from the balloon through a fluid conveyance so as to substantially deflate the balloon, and withdrawing the fluid conveyance without removing the balloon from the perirectal space.

Embodiment 9 is the method of embodiment 8, wherein the balloon is biodegradable in situ.

Embodiment 10 is the method of any one of embodiments 1-9, additionally comprising: aspirating the thermal fluid from the balloon so as to substantially deflate the balloon, and removing the balloon from the perirectal space.

Embodiment 11 is the method of any one of embodiments 1-10, wherein the thermal fluid includes an aqueous saline solution having an NaCl concentration between 0.05 and 0.5 mol/kg.

Embodiment 12 is the method of any one of embodiments 1-11, wherein the reinflating of the balloon is with thermal fluid having a temperature that is between 5° C. and 30° C. warmer than the aspirated fluid, or between 10° C. and 30° C. warmer, or between 10° C. and 20° C. warmer.

Embodiment 13 is the method of any one of embodiments 1-12, wherein the thermal fluid for the reinflation is heated before the reinflation.

Embodiment 14 is the method of any one of embodiments 1-13, wherein a temperature of the aspirated fluid is at least 10° C., or at least 15° C., or at least 20° C.

Embodiment 15 is the method of any one of embodiments 1-14, wherein the temperature of the warmer fluid is at least 20° C. and not more than 42° C.

Embodiment 16 is the method of any one of embodiments 1-15, wherein the circulating is carried out at least twice during the cryoablation therapy session.

Embodiment 17 is the method of any one of embodiments 1-16, wherein the circulating includes at least 2 cycles of aspirating and reinflating during the cryoablation therapy session, or at least 5 cycles, or at least 10 cycles.

Embodiment 18 is the method of any one of embodiments 1-17, wherein the circulating is initiated and/or repeated in response to receiving a temperature measurement of the thermal fluid.

Embodiment 19 is the method of any one of embodiments 1-18, wherein the circulating is initiated and/or repeated in response to receiving a temperature measurement of rectal tissue.

Embodiment 20 is the method of any one of embodiments 18 or 19, wherein the circulating is initiated and/or repeated automatically in response to the temperature measurement.

Embodiment 21 is the method of any one of embodiments 1-20, wherein the circulating is initiated and/or repeated according to a programmed schedule.

Embodiment 22 is the method of any one of embodiments 1-21, wherein at least one aspiration of the cooled thermal fluid is a partial aspiration, and a respective subsequent reinflation is from a state of partial inflation.

Embodiment 23 is the method of embodiment 22, wherein the state of partial inflation is between 10% and 80% by volume of full inflation, or between 40% and 70% by volume of full inflation.

Embodiment 24 is the method of any one of embodiments 1-23, wherein an average temperature of the thermal fluid in the balloon does not go lower than 14° C., or lower than 15° C., or lower than 16° C., or lower than 17° C., or lower than 18° C., or lower than 19° C., or lower than 20° C., or lower than 21° C., or lower than 22° C., or lower than 23° C., or lower than 24° C., during the cryoablation therapy session.

Embodiment 25 is the method of any one of embodiments 1-24, wherein a temperature of rectal tissue in contact with the balloon in an inflated state does not go below 14° C., or below 15° C., or lower than 16° C., or lower than 17° C., or lower than 18° C., or lower than 19° C., or lower than 20° C., or lower than 21° C., or lower than 22° C., or lower than 23° C., or lower than 24° C., during the cryoablation therapy session.

Embodiment 26 is an apparatus for use in safeguarding a subject's rectum during a cryoablation therapy session targeting the subject's prostate, the apparatus comprising:
an inflatable spacer balloon configured to hold a quantity of a thermal fluid and to undergo multiple in situ inflation-deflation cycles; and
a circulation assembly configured to be placed in fluid communication with the balloon and adapted to circulate the thermal fluid by aspirating cooled thermal fluid from the balloon and reinflating the balloon with thermal fluid that is warmer than the aspirated fluid.

Embodiment 27 is the apparatus of embodiment 26, additionally comprising a medical dilator assembly for an ultrasound-guided tissue dissection, the dilator assembly including a lumen for passing the balloon therethrough, in an uninflated state, into a perirectal space between the prostate and the rectum.

Embodiment 28 is the apparatus of any one of embodiments 26 or 27, wherein the circulation assembly comprises a plurality of fluid conveyances, wherein a first fluid conveyance is for aspiration and a second fluid conveyance is for reinflation.

Embodiment 29 is the apparatus of any one of embodiments 26 or 27, wherein the circulation assembly comprises a fluid conveyance in fluid communication with multiple other fluid conveyances.

Embodiment 30 is the apparatus of any one of embodiments 26 or 27, wherein the circulation assembly comprises a fluid conveyance comprising multiple internal lumens, wherein a first lumen is for aspiration and a second lumen is for reinflation.

Embodiment 31 is the apparatus of any one of embodiments 26-28, wherein the circulation assembly comprises a plurality of fluid conveyances, wherein a first fluid conveyance is configured to convey the thermal fluid at a pressure above an ambient pressure, and a second fluid conveyance is configured to convey the thermal fluid at a pressure below the ambient pressure.

Embodiment 32 is the apparatus of embodiment 31, wherein the circulation assembly comprises a fluid conveyance comprising multiple internal lumens, wherein a first lumen is configured to convey the thermal fluid at a pressure above an ambient pressure, and a second fluid lumen is configured to convey the thermal fluid at a pressure below the ambient pressure.

Embodiment 33 is the apparatus of any one of embodiments 26-32, wherein the circulation assembly additionally comprises a temperature sensor for measuring an external temperature of the balloon.

Embodiment 34 is the apparatus of any one of embodiments 26-33, wherein the circulation assembly additionally comprises a temperature sensor for measuring a temperature of thermal fluid within the balloon.

Embodiment 35 is the apparatus of any one of embodiments 26-34, wherein the circulation assembly additionally comprises a temperature sensor for measuring a temperature of thermal fluid aspirated from the balloon.

Embodiment 36 is the apparatus of any one of embodiments 26-35, wherein the circulation assembly additionally comprises a temperature sensor for measuring a temperature of a wall of the rectum.

Embodiment 37 is the apparatus of any one of embodiments 26-36, wherein the circulation assembly additionally comprises a temperature sensor in fluid communication with the heated thermal fluid.

Embodiment 38 is the apparatus of any one of embodiments 26-37, wherein the circulation assembly additionally comprises a circulation controller programmed to initiate a circulating of the thermal fluid in response to a temperature measurement.

Embodiment 39 is the apparatus of any one of embodiments 26-38, wherein the circulation assembly additionally comprises a circulation controller programmed to initiate a circulating of the thermal fluid based on a programmed schedule.

Embodiment 40 is the apparatus of any one of embodiments 26-39, wherein the circulation assembly additionally comprises a circulation controller programmed to initiate a circulating of the thermal fluid based on avoiding exposing the rectum to a temperature below 20° C., or below 15° C., or below 10° C.

Embodiment 41 is the apparatus of any one of embodiments 26-40, wherein the balloon is biodegradable in situ.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art, which would occur to persons skilled in the art upon reading the foregoing description.

1. A method of safeguarding a subject's rectum during a cryoablation therapy session targeting the subject's prostate, the method comprising:
inserting a deflated tissue-dissection balloon into a perirectal space between the prostate and the rectum;
inflating the balloon with a thermal fluid so as to create a thermal-separation barrier between the prostate and a rectal wall; and
circulating the thermal fluid by aspirating, from the balloon, thermal fluid cooled by the targeted cryoablation therapy, and reinflating the balloon with thermal fluid that is warmer than the aspirated fluid.

2. The method of claim 1, wherein the inserting is carried out subsequent to an introduction of one or more cryoprobes to the prostate.

3. The method of claim 1, wherein the circulating is carried out subsequent to introduction of one or more cryoprobes to the prostate.

4-7. (canceled)

8. The method of claim 1, additionally comprising: aspirating the thermal fluid from the balloon through a fluid conveyance so as to substantially deflate the balloon, and withdrawing the fluid conveyance without removing the balloon from the perirectal space.

9-27. (canceled)

28. An apparatus for use in safeguarding a subject's rectum during a cryoablation therapy session targeting the subject's prostate, the apparatus comprising:
an inflatable spacer balloon configured to hold a quantity of a thermal fluid and to undergo multiple in situ inflation-deflation cycles; and
a circulation assembly configured to be placed in fluid communication with the balloon and adapted to circulate the thermal fluid by aspirating cooled thermal fluid from the balloon and reinflating the balloon with thermal fluid that is warmer than the aspirated fluid.

29. The apparatus of claim 28, additionally comprising a medical dilator assembly for an ultrasound-guided tissue dissection, the dilator assembly including a lumen for passing the balloon therethrough, in an uninflated state, into a perirectal space between the prostate and the rectum.

30. The apparatus of claim 28, wherein the circulation assembly comprises a plurality of fluid conveyances, wherein a first fluid conveyance is for aspiration and a second fluid conveyance is for reinflation.

31. The apparatus of claim 28, wherein the circulation assembly comprises a plurality of fluid conveyances, wherein a first fluid conveyance is configured to convey the thermal fluid at a pressure above an ambient pressure, and a second fluid conveyance is configured to convey the thermal fluid at a pressure below the ambient pressure.

32. The apparatus of claim 28, wherein the circulation assembly comprises a fluid conveyance in fluid communication with multiple other fluid conveyances.

33. The apparatus of claim 28, wherein the circulation assembly comprises a fluid conveyance comprising multiple internal lumens, wherein a first lumen is for aspiration and a second lumen is for reinflation.

34. The apparatus of claim 33, wherein the circulation assembly comprises a fluid conveyance comprising multiple internal lumens, wherein a first lumen is configured to convey the thermal fluid at a pressure above an ambient pressure, and a second fluid lumen is configured to convey the thermal fluid at a pressure below the ambient pressure.

35. The apparatus of claim 28, wherein the circulation assembly additionally comprises a temperature sensor for measuring an external temperature of the balloon.

36. The apparatus of claim 28, wherein the circulation assembly additionally comprises a temperature sensor for measuring a temperature of thermal fluid within the balloon.

37. The apparatus of claim 28, wherein the circulation assembly additionally comprises a temperature sensor for measuring a temperature of thermal fluid aspirated from the balloon.

38. The apparatus of claim 28, wherein the circulation assembly additionally comprises a temperature sensor for measuring a temperature of a wall of the rectum.

39. The apparatus of claim 28, wherein the circulation assembly additionally comprises a temperature sensor in fluid communication with the heated thermal fluid.

40. The apparatus of claim 28, wherein the circulation assembly additionally comprises a circulation controller programmed to initiate a circulating of the thermal fluid in response to a temperature measurement.

41. The apparatus of claim 28, wherein the circulation assembly additionally comprises a circulation controller programmed to initiate a circulating of the thermal fluid based on a programmed schedule.

42. The apparatus of claim 28, wherein the circulation assembly additionally comprises a circulation controller programmed to initiate a circulating of the thermal fluid based on avoiding exposing the rectum to a temperature below 20° C., or below 15° C., or below 10° C.

43. The apparatus of claim 28, wherein the balloon is biodegradable in situ.
